(19) United States
(12) Patent Application Publication
(10) Pub. No.: US 2001/0051798 A1
(43) Pub. Date: Dec. 13, 2001

(54) Method of performing an injection using a bi-directional rotational insertion technique

(76) Inventor: Mark N. Hochman, Lake Success, NY (US)

Correspondence Address: GOTTLIEB RACKMAN & REISMAN PC, 270 MADISON AVENUE, 8TH FLOOR, NEW YORK, NY, 100160601

(21) Appl. No.: 09/745,751

(22) Filed: Dec. 21, 2000

Related U.S. Application Data
(60) Provisional application No. 60/173,374, filed on Dec. 28, 1999.

(51) Int. Cl. A61M 31/00

(52) U.S. Cl. 604/506; 604/218

(57) ABSTRACT

A drug is administered to a patient through a beveled needle by rotating the needle as it is advanced into tissues. The rotation of the needle insures that the needle is not deflected as it is advanced. In this manner, the amount of pain felt by the patient may be reduced, and the drug is delivered to accurately to the selected site.

### BACKGROUND OF THE INVENTION

1. Field of Invention

This invention pertains to a novel method of performing an injection by a doctor, nurse and other health practitioner. More particularly, the invention pertains to a method for delivering an injection wherein the needle of the injection apparatus is simultaneously rotated and translated to reduce pain in the patient and to eliminate undesirable needle deflections.

2. Description of the Prior Art

The notion that a hollow core needle could be used to inject a local anesthetic solution into the body was unknown until the late 1800's. When an American surgeon Dr. William Halstead demonstrated that an interstitial injection of aqueous cocaine resulted in an effective inferior alveolar nerve block he ushered in a new era of local pain management for both dentistry and medicine. Since that time, numerous improvements in the safety and efficacy of local anesthesia have evolved. The majority of these advancements have been related to the pharmacology and formulation of anesthetic agents making local pain control safer and more effective. In contrast, improvements to the drug delivery device (i.e. hypodermic syringe) have been few. The introduction of the manual aspirating syringe used in dentistry today has actually made the instrument less ergonomic for the operator to use than the non-aspirating version. Much advancement has been made in needle design over the past century. The development of a disposable needle had a major impact on all syringe injections because it insured sterility as well as consistent sharpness. Further advancements in metallurgy, surface treatments and manufacturing techniques have resulted in modern needles of unparalleled sharpness. Presumably, a sharper needle penetrates body tissues more easily thus resulting in less discomfort for the patient.

The use of a hypodermic needle in dentistry (as well as other medical fields) has been consistently shown to produce a deflection if an eccentric pointed cylindrical hypodermic needle is used( See Aldous J. Needle Deflection: a factor in the administration of local anesthetics. JADA 1968;77:602-04. Robinson SF, Mayhew, Cowan RD, Hawley RJ. Comparative study of deflection characteristics and fragility of 25-, 27-, and 30-gauge dentalneedls. JADA 1984;109:920-24.).

Successful local anesthesia is critical to the daily practice of dentistry. It is a prerequisite to insure maximum patient comfort while performing a wide variety of clinical procedures on the hard and soft tissues of the oral cavity. Therefore, achieving predictable results in local anesthesia is of great importance to all clinicians. Failure to do so can lead to increased stress for both the operator and the patient. An injection that is recognized as one of the more difficult in dentistry is the inferior alveolar (IA) nerve block. There are a number of physical factors that have been associated with the relative success or failure of the IA nerve block. They include anatomical variations between patients, operator technique and needle deflection.

Contemporary dental anesthesia textbooks attribute needle deflection as a source of anesthesia failures. It has been reported that the IA rate of failure can range from 20% to 30% and most dentists have experienced some difficulty with this injection. The inferior alveolar nerve is contained within the pterygoman-dibular space. For a needle tip to be in close proximity to the intended target, it must penetrate a variety of tissue types including mucosa, buccinator muscle, submucosal connective tissue, fat and the temporopterygoid fascia.

The needle initiates its path when it first enters through the buccal mucosa at a point between the pterygomandibular raphe and temporal crest of the mandible ramus. The mucosa should be held firmly in place during insertion for precise needle entry. The standard technique requires needle penetration of the buccinator muscle and fascia. As the needle advances it will traverse the connective tissue and adipose tissue found within the pterygomandibular space. The final intended target for the needle is the mandibular foramen found distal and inferior to the mandible lingula . All these tissue layers offer varying degrees of resistance to needle penetration. The entire inferior alveolar neurovascular bundle has a diameter of approximately 2.2 mm, and the pterygomandibular space has a total estimated volume of only 2 cc. Deviation from the final intended target, no matter how small, may have a negative effect on the success of an IA nerve block.

It has long been suggested that all needles deflect irrespective of the diameter of the needle being used. Aldous (identified above) was the first to devise a dynamic testing method to record deflection and he concluded that needle deflection was inversely related to needle diameter.

Robinson(identified above) investigated deflection modifying Aldous's model to improve the measuring and recording accuracy. Robinson concluded that all the needles tested deflected irrespective of gauge. Robinson stated that the degree to which needles deflect is not related to diameter shaft, but maybe more related to the specific metals used in manufacturing.

A previous study has shown that bevel tip design of a needle will influence the path the needle takes as it penetrates through substances of varying densities. It is apparent that a force system is produced on the needle bevel surface. This force vector system is the same for any cylindrical object with a beveled end and it will follow Newton's third physical law of equal and opposite forces. Therefore, an application of a resultant vector force on the beveled surface of an eccentric pointed cylindrical shaft will produce physical bending (deflection) along the path of insertion as illustrated in more detail below. The amount of deflection exhibited by the beveled cylindrical object is determined by the sum of the forces acting on an object in a specific medium.

A bi-beveled needle has the advantage of possessing a needle tip that is centrally located along the needle shaft. Testing this needle design yielded the expected results of reduced needle shaft deflection. The bi-beveled needle eliminates the perpendicular forces that are responsible for needle shaft deflection. However, the most common needle commercially available is an eccentrically pointed beveled needle. Another novel needle is the Accujet® needle (Astra Pharm., Wayne, Pa). This needle enables bevel orientation to be monitored. A visual marker on the needle hub allows the operator to position the bevel in a specific direction. It is thought that this will assist the dentist in better control to the final needle position. The needles listed above require the operator to use a linear insertion technique.

Berns and Sadove conducted a radiograhic in-vivo study. Sixty-six IA nerve block injections were performed on adult patients using a 22-gauge needle administering a mixture of local anesthestic and radiopaque dye. Cephalometric lateral head films were taken with the needle inserted to the proper depth, and securely positioned in place. Review of the reproduced radiographic images appearing in the article demonstrates needle bending with a rigid 22-gauge needle at its final position. The authors stated that the needle tip should be no more than 0.5 cm from the mandibular foramen. They concluded the closer the needle tip placement to the mandibular foramen, the more likely the success of the IA nerve block. The study's conclusion supports the observation that there is a direct correlation between a positive clinical outcome, i.e. anesthesia and the positioning of the needle tip. The study documents radiographic evidence of in-vivo needle deflection. It is therefore not unreasonable to infer that needle deflection affects final needle tip position thus affecting clinical success.

Needle deflection (i.e. bending) is also know to be a contributing factor to inaccurate needle placement and reduced success of injection techniques (Jasktak JT, Yagiela JA, Donaldson D. Local Anesthesia of the Oral Cavity. Philiadephia: WB Saunders Co; 1995. Malamed S. Handbook of Local Anesthsia. 4^{th} Ed. St. Louis: Mosby; 1997.) Currently there are no known techniques available that enable the user to provide an injection with an eccentric pointed hollow core needle in a manner with reduces or eliminates needle deflection and its undesirable side effects.

Existing needle device are known which incorporate rotating mechanism however these were designed specifically for drilling through bony tissues and do not use rely on, nor do they provide a high tactile control during use.

To summarize, all of the above-described prior art have either one or more of the following deficiencies. They describe needle insertion techniques that are cumbersome and do not provide for or even recognize the advantages of using a bidirectional rotational technique for administering injections. Existing devices are cumbersome to perform. Exiting syringes and the like are not designed to allow the operator to use a bi-rotational insertion technique for entry and removal. Exiting syringes and the like are not designed to allow the operator to use a rotational insertion technique for entry and removal

### OBJECTIVES AND SUMMARY OF THE INVENTION

The proposed invention has been designed to reduce or eliminate the undesirable effect of needle deflection. In addition the proposed invention has been designed to reduce the force required during needle penetration and insertion of an eccentric pointed hollow core hypodermic needle.

An objective of the present invention is to provide a technique or method which can be used to provide injections in a manner selected to reduce or eliminate the undesirable effect of needle deflection.

A further objective is to provide a method adapted to reduce the force required during needle penetration and insertion of an eccentric pointed hollow core hypodermic needle.

The subject invention pertains to a novel needle insertion technique designed to overcome the undesirable effect of needle deflection. This technique seeks to produce a more accurate, linear needle tracking through substances regardless of needle gauge. In one embodiment of the invention, the technique relies on a pen-like grasp that makes it possible to rotate a needle in a back-and-forth manner. The needle is rotated between the thumb and index finger 180 degrees in each direction. The type of rotation used is analogous to techniques that have been described for endodontic file instrumentation and acupuncture, however, those techniques no fluid is injected from a needle. More importantly in these latter techniques a needle is first inserted linearly into a tissue and then rotated.

The purpose of the bi-directional rotation is to neutralize the force vectors that act on the needle bevel that make the needle shaft bend. This bi-directional rotation action is preferably maintained during the entire course of needle advancement In order to validate the technique, a study has been performed to test the bending of needles under various conditions. During this testing a protocol for the study followed the design set forth by Robinson (identified above).

Three deflection test models were constructed. The test models differed in the tissue-like substances that were used. In each of the three models, the needle was inserted to a depth of 20 mm. This standardized working length was selected on the availability of a 30-gauge 1 inch (25.4 mm) needle.

These tests have shown that use of the bi-directional rotation insertion technique, even with an eccentric-point bevel needle, allows the operator to cancel-out the perpendicular force vectors on the bevel that cause bending along the needle shaft. The technique generates resultant forces that promote the needle to travel in a linear path. The straight path produced by the bi-directional rotational insertion technique occurs irrespective of needle gauge, bevel design or the metal alloys used in manufacturing.

The present inventor has further discovered that needle deflection requires increased penetration force during the administration of an injection. It is believed that this increased penetration force results in increased and unnecessary tissue damage as well.

### BRIEF DESCRIPTION OF THE DRAWINGS

Fig. 1A shows how a standard injection syringe is held;

FIG. 1B shows how a Wand-type injection handle is held;

FIG. 2A shows the force vector system on a needle during a standard linear insertion technique;

FIG. 2B shows the force vector system on a needle during the inventive bi-directional insertion system;

FIG. 3A and 3B each show typical deflections for needles inserted using a standard linear technique as opposed to a bi-directional technique, the two graphs being taken orthogonally with respect to each other;

FIG. 4 shows a chart for the range, average and standard deviation of the deflections for a 30, 27 and 25 gauge needle ,using the standard and the bidirectional insertion techniques.

### DETAILED DESCRIPTION OF THE INVENTION

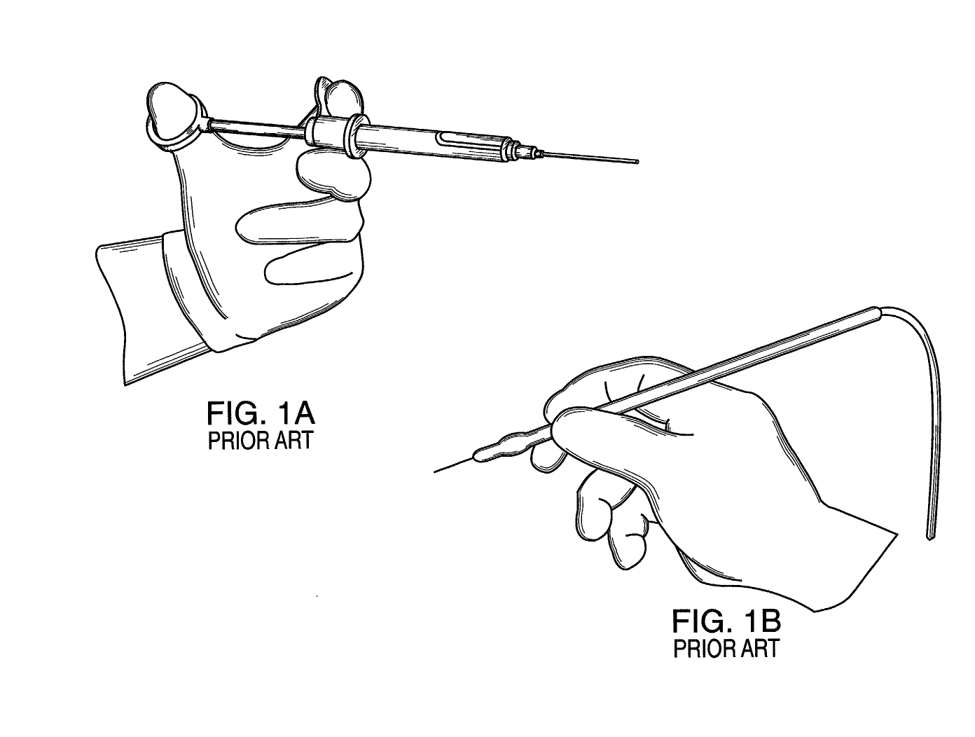

FIGS. 1A and 1B show two different means by which an injection may be performed using a standard linear insertion technique. FIG. 1A shows a standard palm/thumb grasp used on a syringe with a needle (usually having a beveled tip-not shown in the Figure). FIG. 1B shows a pen grasp for holding a handle terminating with a needle. The handle may be part of an automatic injection pump such as the WANDS ® available from Milestone Scientific Corporation of Livingstone, New Jersey.

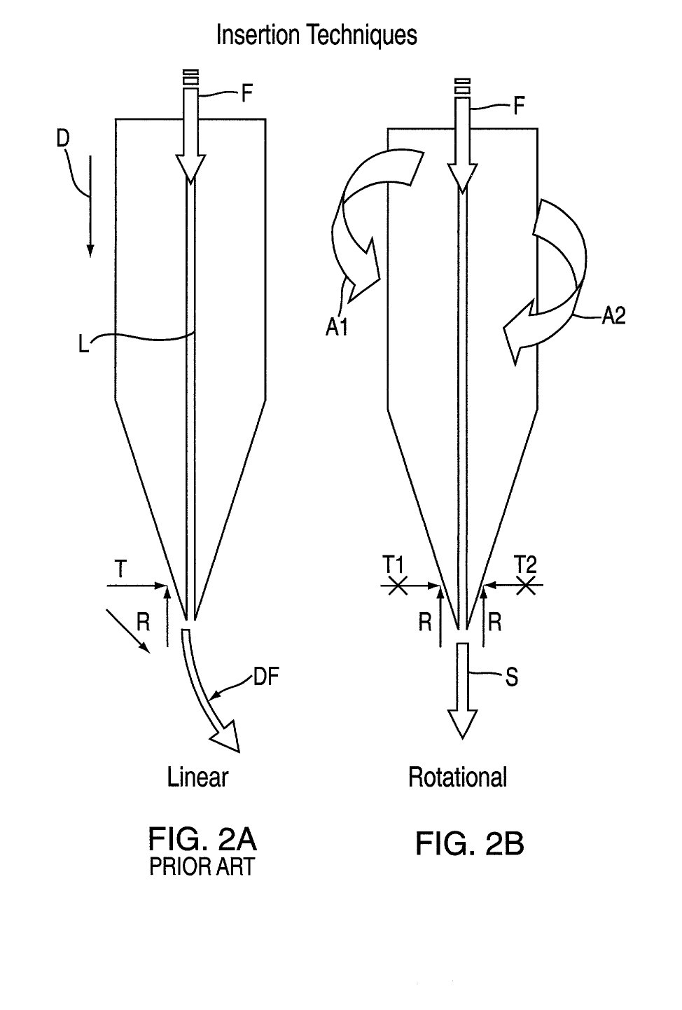

The present inventor has discovered that all the problems associated with injections discussed above can be eliminated with a novel bidirectional injection technique. The proposed technique and its advantages are best understood by reviewing the somewhat diagrammatic illustrations of FIGS. 2A and 2B. In FIG. 2A a needle N having a lumen L is advanced linearly (using the grasp of FIG. 1A or 1B, for example) in the direction indicated by arrow D while a fluid F is being injected through the lumen L. The advancement of the needle is resisted by the force R generated by the tissues (not shown) and because of the beveling of the needle, a transversal force T is generated which causes the needle N to bend or deflect as indicated by the arrow DF. However, if the needle is rotated first in one direction A1 and then in a second direction A2, the effects of transversal forces T1, T2 cancel and are neutralized, or, at least, minimized causing the needle to be inserted in a relatively straight manner, as indicated by arrow S.

The amount of rotation to be imparted to the needle depends at least to some extent on the amount of its longitudinal travel, which in turn depends on the depth within the tissue at which a drug needs to be and the speed at which the needle is advanced. Typically, the needle is advanced at about 2-4 mm/sec. For a shallow depth of about 2-4 mm, the total rotation imparted to the needle may be relatively small. For example, the needle may be rotated by 180 degrees in one direction and 180 degrees in the other. For longer travel distances, the needle may be rotated in several cycles, each cycle comprising rotating the needle by an angle A and then rotating the needle in the opposite direction by the same angle A. As discussed above, preferably A is 180 degrees although it may be other values as well. Moreover, the needle need not be rotated by the same angle A each time, and need not be returned to the same angular position. Similar effects may be obtained if, instead of rotating the needle back and forth in two directions, it is continuously rotated in a single direction over, for example, 360 degrees.

The traditional handheld syringe requires a palm-thumb grasp (FIG. 1A) and does not lend itself easily to the rotational insertion technique. This may explain why the technique has not been described in the past. However the recently introduced anesthetic delivery system (The WandTM, Milestone Scientific, Inc., Livingstone, Nj) illustrated in FIG. 1B was designed to use a lightweight, disposable pen-like handpiece requiring the operator to use a thumb and index finger grasp. The benefits of a bidirectional rotation insertion technique can be maximized with this pen-like grasp.

Thus the bidirectional rotational movement of the needle may be accomplished either manually or automatically. If a handle is used to administer an injection, as shown in FIG. 1B then the needle can be rotated back and forth easily by 180 degrees (or any other angle) by merely rotating the handle as the needle is advanced. Alternatively, the needle may be rotated automatically as it advances, as it is disclosed in commonly assigned co-pending application Ser. No. 506,484 filed Feb. 17, 2000 entitled A HAND-PIECE FOR INJECTION DEVICE WITH A RETRACTABLE AND ROTATING NEEDLE and incorporated herein by reference. This application discloses a needle which is normally disposed in a housing to protect health practitioners from being pricked. The needle can be selectively advanced in a longitudinal direction so that it can extend outwardly of the housing. In one embodiment, the needle rest on a support which includes an extension engaging a helical track inside the housing. As the needle is advanced and retracted, the extension rides in the helical track in a caming action causing the needle to rotate in a first direction and then in a second direction.

In order to validate this concept, a rigorous set of in vitro tests have been conducted to study needle penetration and deflections. The most widely accepted model for studying needle deflection is an in-vitro model utilizing tissue-like substances. This type of experimentation provides a reliable testing environment without the need for human tissues and eliminates many of the difficult ethical questions raised by animal studies. It is known that this type of testing provides valuable insight into needle characteristics in an experimental setting.

Early studies have shown that needle diameter (gauge) and the relative flexibility or resilience of the needle shaft are some of the physical characteristics reported to affect needle deflection. These early studies have also concluded that shaft diameter is the most critical factor affecting bending or deflection of the needle.

Controversy in the literature exists regarding the factors responsible for needle deflection. The inventor has conducted a study to determine if using a new bi-directional rotation insertion technique could minimize needle deflection.

Testing Methods and Materials

Three deflection test models were constructed. The test models differed in the substances used to simulate tissues. In each of the three models, the needle was inserted to a depth of 20 mm. This standardized working length was selected on the availability of a 30-gauge 1 inch (25.4 mm) needle. The following materials served to simulate tissues: hydrocolloid (test material A), frankfurters (test material B), and soft bite wafer wax (test material C). These test materials have various densities to simulate various types of tissues.

All three tests employed a modified dental surveyor (Ney Co., Chicago, Il.) to produce standardized needle insertions. For each material three different size needle gauges were tested: a 30-gauge 1 inch needle; a 27-gauge and a 25-gauge needle, the last two needles being 1 ¼ inch long (MonojetUltra ® Sharp Model 400, Sherwood Medical Co., St. Louis, Mo). Traditional Luer type connectors were attached to a customized arm of the surveyor. The needle was then advanced into each material using either the transitional linear or the bidirectional rotation insertion technique. A sufficient number of tests were performed for each needle within a substance to provide for adequate statistical relevance.

Tests Using Material A

A hydrocolloid material (Acculoidl™ Extra Strength, Van R Dental Products, Inc. Product #11110) was placed into a 6-oz. plastic container which fit into the custom surveyor jig. The jig was constructed to produce consistent, perpendicular orientation of the x-ray tube head. The custom jig was designed to record needle deflection in orthogonal two planes. This enabled the total amount of deflection to be determinable from a simple algebraic formula. A total of 60 insertions were performed using 30 needles (10 needles for each needle gauge size).

Each needle served as its own control between the two techniques. The needle was first inserted into the tissue-like substance with a linear non-rotating movement. The same needle was then inserted into the test material using the bi-directional rotation insertion technique. After the needle was used for the second insertion technique it was discarded and the test was repeated using a new needle.

After each needle insertion two x-ray films were exposed at 15MA, 65 KVP, 10 impulses and then developed. A metallic x-ray grid was used to record the maximum amount of deflection produced. Each film was measured with a Boley gauge on a superimposed grid from the point of insertion to the tip of the needle. The total amount of deflection produced was calculated using a geometric principle as described by Robinson.

Tests On Material B

Deflection test material-B was a processed precooked meat-namely, frankfurters (Hebrew National, Inc., Bronx, Ny). The identical protocol of the test for material A was followed. A total of 42 insertions were performed using

Rotatinal Insertion Technique 21 needles (7 needles for each needle gauge size, 30, 27 and 25-gauge).

Tests On Material C

Material C was made of a soft wax bite-wafer (The Hygenic Corp. Akron, Oh). A custom platform was constructed which aligns the wax parallel to the long axis of the needle held by the dental surveyor arm. The use of soft wax bite-wafer allowed visual inspection to measure and determine the amount of needle deflection observed.

Orientation of the needle bevel was perpendicular to the surface of the wax, and this was confirmed by the operator wearing 2.5x magnification loops (Designs for Vision, Inc. Ronkonkoma, Ny). The needle was first inserted to a depth of 20 mm into the wax using a non-rotational linear movement. Marking the wax at a point were the needle tip ended in the wax identified the deflection. The needle was removed from the wax and positioned in front with the needle shaft aligned to the access hole created from the initial insertion. A Boley gauge was used to measure the distance of deflection that was observed. The same needle was employed for the second test, the bi-directional rotation insertion technique. Each needle therefore served as its own control. A total of 100 insertions were performed using 50 needles of a 30-gauge size. An additional 40 insertions using 10 needles each of 27 and 25-gauge was conducted to compare the two techniques. The needles used for this study were randomly selected from a standard box of 1100 needles as supplied by a local dental distributor.

Results

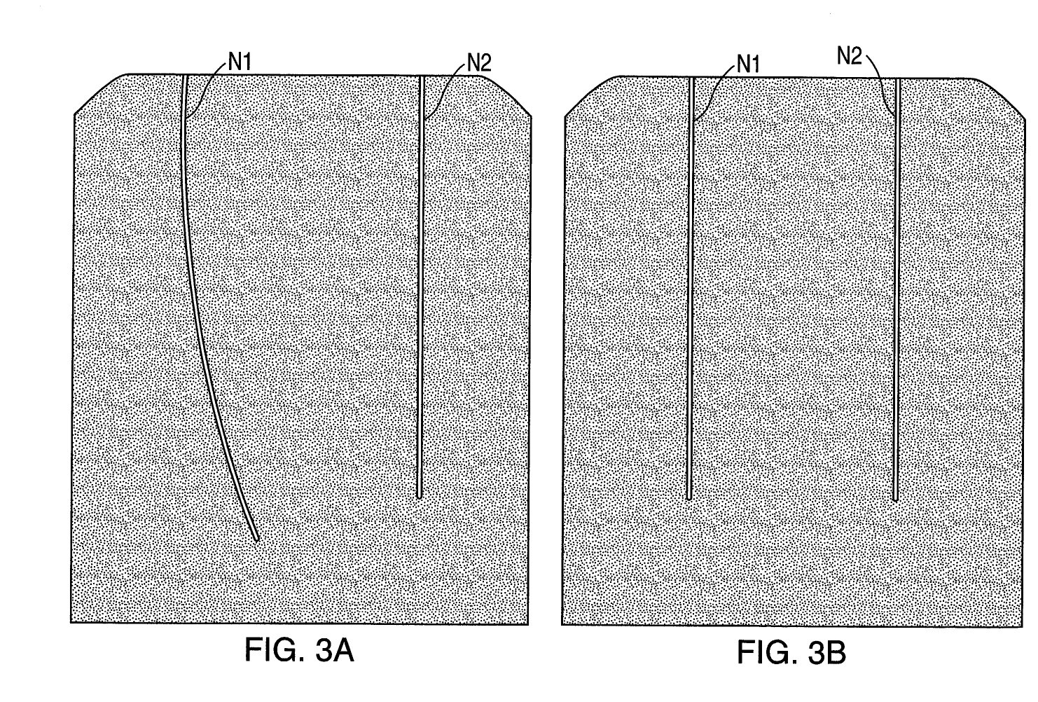

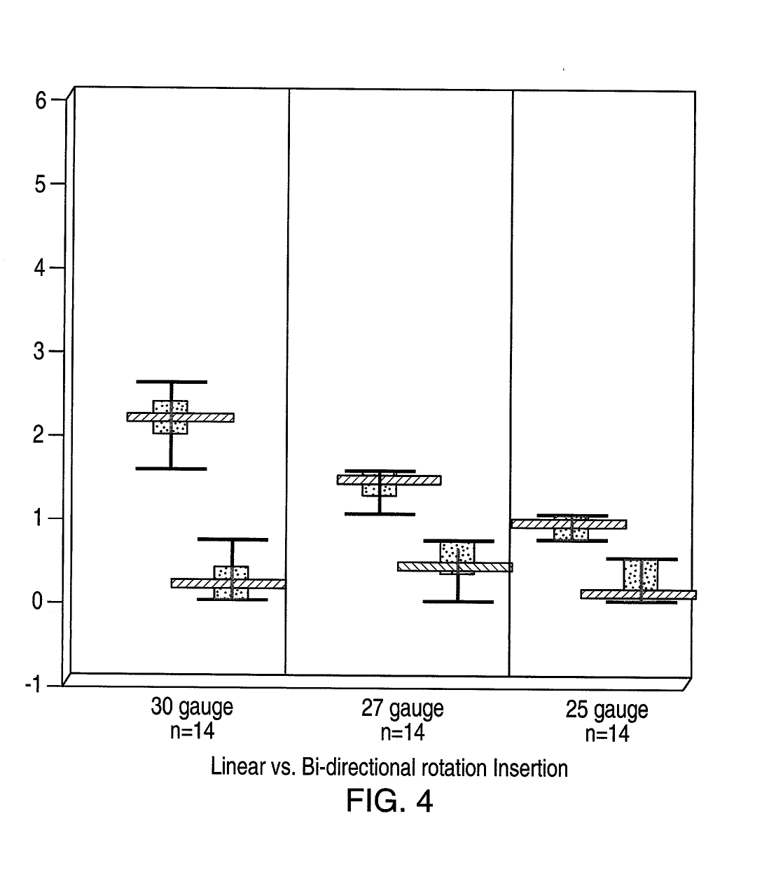

FIGS. 3A and 3B show typical results of these tests. More specifically, in FIG. 3A, needle N1 was inserted using a standard linear technique and needle N2 was inserted using the subject bidirectional rotational technique. The large amount of deflection caused by the standard linear technique when compared to the deflection of needle N2 is clearly visible in this Figure. In FIG. 3B taken orthogonally to FIG. 3A, virtually no deflection for either needles N1, N2 is seen because of the way the two sets of radiographs have been selected so that maximum deflection (as determined by the beveling of the needles) is visible in FIG. 3A.

Statistical data analysis was performed by paired T-tests for each experiment. The rotational technique described was consistently more effective in minimizing and eliminating needle shaft deflection for a 30-gauge, 27-gauge and 25-gauge needle. Each of the different tissue-like substances tested consistently demonstrated this reduction in needle deflection with the bi-directional rotation insertion technique.

Differences in deflection between linear and rotational insertion were found to be statistically Insignificant (P<.05) in each of the experiments conducted. A 95% confidence level with no overlap of the upper and lower limits was observed.

When comparing linear insertion to bidirectional rotation insertion, the mean amount of total deflection of a 30-gauge needle in wax was 2.7 mm vs. 0.1 mm, respectively. In hydrocolloid, the total mean deflection was 4.7 mm vs. 1.1 mm comparing linear to rotational insertion. In frankfurters, the total mean deflection between linear and rotational insertion was 2.2 mm vs. 0.2 mm.

The comparison of linear to bi-directional rotation insertion technique for a 27-gauge needle was as follows: total mean deflection in wax was 3.4 mm vs. 0.1 mm, in hydrocolloid was 4.6 mm vs. 0.8 mm, in frankfurter was 1.4 mm vs. 0.6 mm respectively.

The comparison of linear to bi-directional rotation insertion technique for a 25-gauge needle was as follows: total mean deflection in wax was 2.6 mm vs. 0.1 mm; in hydrocolloid 3.8 mm vs. 0.5 mm; in frankfurter 0.9 mm vs. 0.2 mm respectively.

In addition, the bi-directional rotational insertion technique also reduces substantially the force required to push the needle to penetrate tissues. Preliminary data suggests that a reduction of force penetration in the range of 40% to 50% can be anticipated when using of this technique. This may prove to be particularly beneficial for those injections that penetrate dense connective tissue, i.e., palatal tissue of the oral cavity.

The density of the substance that a needle is inserted into appears to influence the amount of deflection produced by the bevel. Tissue-like substances with greater density, i.e., hydrocolloid, consistently produced greater deflection compared with less dense substances. Encountering a fluid filled compartment would minimize deflection relative to the fluid viscosity. The oral cavity is primarily composed of tissues with a spectrum of varied densities. These densities fall within a broad range.

In the testing model, it was critical to provide a consistent and uniform material to eliminate variations between samples. A variety of different types of materials were tested reflecting a range of different densities. There are no published studies available that quantify densities of oral tissues in the infratemporal fossa. The materials selected offered a reasonable spectrum that is analogous to tissues that might be encountered. It is apparent that the type of insertion technique used had the greatest influence on the amount of deflection produced irrespective of the density of the substance tested.

Needle length appears to be another factor that influences the amount of deflection. The standard testing distance of 20 mm was selected in this study based on the commercial availability of a 30-gauge, 1 inch needle. It is noted that insertion distances of 25 mm and more are typical for the IA nerve block. It would be expected that these greater distances would reflect greater rates of deflection. Longer needles that travel greater distances will demonstrate larger amounts of bending then those observed in this study. This would only accentuate this study's finding.

The increased length of the thicker needle can explain the finding of increased needle deflection of 27-gauge needles compared to 30-gauge needles in the denser tissue-like substance (wax). The standard 27-gauge needle is ¼ inch (6mm) longer than the 30-gauge needle producing increased “springiness”. This could account for the greater bending of the needle that is observed. Irrespective of differences between the different needle sizes, all needles demonstrated a significant reduction in deflection with the bi-directional rotation insertion technique.

The study design always tested linear insertion followed by rotational insertion. Maintaining this order of needle insertions was believed to minimize bias produced from a dulling or deforming of the needle.

This study has demonstrated that a needle that traverses 20 mm of a tissue-like substance can deflect as much as 5 mm. The bi-direction rotation insertion technique provides greater accuracy of placement for those injections that require deep needle penetration.

For injections in the palate or other supraperiosteal infiltration injections, high-level accuracy may not be necessary to achieve successful anesthesia. However, it was noted that all needle penetrations required reduced force when the bi-directional rotation technique is used. This suggests that the needle penetration force may be reduced by the rotational insertion technique.

Conclusion

The success of local anesthesia in dentistry is multi-factorial. One of the most challenging of all local anesthesia injections is the inferior alveolar nerve block. Not all anesthetic failures are related to needle deflection. However, needle deflection has been identified as one of the elements that can reduce the accuracy and predictably of the IA nerve block. This study was conducted to investigate the cause and effect relationship between the needle and deflection.

The factor that most greatly affects the path taken through a tissue-like substance by an eccentric beveled needle is the force vectors that act upon the beveled surface.

The use of a bi-directional rotation insertion technique minimizes needle deflection, resulting in a straighter tracking path for the 30-, 27- and 25-gauge dental needles.

The use of a bidirectional rotation insertion technique minimizes needle deflection in the three different tissue-like substances tested in this study.

Modifications may be to the invention described herein without departing from its scope as defined in the appended claims.

I claim:

1. A method of injecting a drug into a patient through a needle having a lumen comprising the steps of:
advancing said needle into the tissue linearly along a longitudinal axis of the needle; and
simultaneously rotating the needle along its longitudinal axis to reduce deflection of the needle.

2. The method of claim I wherein said needle is rotated for an angle of about 180 degrees.

3. The method of claim 1 wherein said simultaneous rotation is a bidirectional rotation whereby the needle is rotated in a first direction and then in a second direction.

4. The method of claim 3 wherein the needle is returned to its original angular orientation after each rotation.

5. The method of claim 3 wherein said rotation comprises rotating the needle by an angle of 0-180 degrees.

6. The method of claim 5 wherein said needle is advanced at a rate of 2-4 mm/sec during said rotation.

7. A method of administering drug to a patient comprising the steps of:
providing a needle associated with a drug supply, said needle having an elongated shaft, a lumen and a beveled tip with an exit point communicating with said lumen so that said drug is forced from said drug supply through said lumen and out of said exit point;
advancing said needle along a longitudinal axis of the needle through the patient tissue until a predetermined site is reached; and
simultaneously rotating said needle about said longitudinal axis during said advancing to prevent said needle from being deflected.

8. The method of claim 7 wherein said rotating includes rotating said needle first on a first direction and then rotating said needle in a second direction opposite said first direction.

9. The method of claim 7 wherein said rotating includes rotating said needle from said first orientation and then returning said needle to said first orientation.

10. The method of claim 9 wherein said needle is rotated by a predetermined angle in a first direction and is then rotated backwards by the same predetermined angel to said first predetermined location.

11. The method of claim 10 wherein said needle is rotated by an angle of between 0-180 degrees.

12. The method of claim 7 wherein said needle is rotated cyclically several times as said needle is advanced.

13. The method of claim 7 wherein said needle is rotated manually.
